(19) United States
(12) Patent Application Publication
(10) Pub. No.: US 2025/0325458 A1
(43) Pub. Date: Oct. 23, 2025

(54) COSMETIC COMPOSITION COMPRISING FERULIC ACID AS ACTIVE INGREDIENT

(71) Applicant: LG H&H CO., LTD., Seoul (KR)

(72) Inventors: Jung Hyuk MOON, Seoul (KR); Hye In WOO, Seoul (KR); Yeon Kyung KIM, Seoul (KR); Nam-Seo SON, Seoul (KR)

(73) Assignee: LG H&H CO., LTD., Seoul (KR)

(21) Appl. No.: 18/870,618

(22) PCT Filed: May 31, 2023
(86) PCT No.: PCT/KR2023/007453

§ 371 Date: Nov. 29, 2024

(30) Foreign Application Priority Data
May 31, 2022 (KR) 10-2022-0066921

(51) Int. Cl. A61K 8/365; A61K 8/34; A61K 8/44; A61K 8/49; A61Q 19/00

(52) U.S. Cl. CPC A61K 8/365; A61K 8/345; A61K 8/44; A61K 8/4946; A61Q 19/00; A61K 2800/52

(57) ABSTRACT

The present invention relates to a cosmetic composition comprising ferulic acid, a basic amino acid, and a pH adjusting agent, wherein decarboxylation reactions of the ferulic acid are suppressed so that the titer of the ferulic acid can be maintained even during long-term storage, and precipitation, discoloration, and deterioration of efficacy are prevented, thus providing a composition having excellent formulation stability.

### TECHNICAL FIELD

The present invention relates to a cosmetic composition including ferulic acid as an active ingredient, and more particularly, to a cosmetic composition including ferulic acid; a basic amino acid; and a pH adjusting agent.

### BACKGROUND ART

Ferulic acid has a wide range of antibacterial activity and is effective in neutralizing reactive oxygen, and as an external preparation for skin, it can exhibit various effects such as antioxidant, UV protection, and skin brightening effects.

Therefore, there have been sustained efforts to develop cosmetics containing ferulic acid.

However, since ferulic acid has very low solubility and low stability, its use in cosmetics has been limited. Specifically, since the pKa of ferulic acid is 4.61, there were concerns that ferulic acid may precipitate at pH 5 or below, and at pH 5 or above, ferulic acid is decomposed due to the decarboxylation reaction of ferulic acid, causing discoloration of the formulation and reductions in the titer and efficacy of ferulic acid.

In order to overcome the problem of the precipitation of ferulic acid at pH 5 or below, attempts have been made to improve solubility and storage stability by adding glycols and non-volatile polyols along with ferulic acid. However, the content of glycols had to increase along with the content of ferulic acid, and there was a risk of skin irritation when using glycols in excess amounts.

Since the pKa of ferulic acid is lower than the pH of human skin, which is 5.5 to 6, there was also a concern about skin irritation. Therefore, in terms of safety for the human body, there was a need to develop products that have a pH similar to the pH of human skin or the pH of common cosmetics while using a small amount of polyol.

At pH 5 or above, the solubility of ferulic acid was improved, reducing the usage amount of polyol and the concern about skin irritation. However, at pH 5 or above, the decarboxylation reaction of ferulic acid occurred, causing rapid discoloration and reductions in the titer and efficacy of the composition, which can cause problems in the distribution of cosmetics.

In addition, an attempt was made to improve the formulation stability of ferulic acid by derivatizing ferulic acid. However, its derivatives improved formulation stability but did not have as much of a skin improvement effect as ferulic acid. Thus, a method was needed to stably contain ferulic acid at a certain amount or more, rather than derivatizing ferulic acid.

### DISCLOSURE

### Technical Problem

Against this background, the present inventors tried to improve the stability of ferulic acid and completed the present invention after confirming that the addition of a basic amino acid and a pH adjusting agent to ferulic acid prevents precipitation of ferulic acid and inhibits the decarboxylative decomposition reaction of ferulic acid, thereby preventing the discoloration and reductions in the titer and efficacy of the composition.

Accordingly, the object of the present invention is to provide a cosmetic composition with excellent formulation stability by preventing the discoloration, precipitation, and reductions in the titer and efficacy of the composition including ferulic acid.

### Technical Solution

The present invention provides a cosmetic composition including ferulic acid, a basic amino acid; and a pH adjusting agent, wherein the content of the ferulic acid is 0.1 wt % or more based on the total weight of the composition, and the composition has a pH of 4.0 to 6.0.

In addition, the present invention provides an external preparation for skin including the composition.

### Advantageous Effects

The present invention can provide a composition with excellent formulation stability by inhibiting the decarboxylative decomposition reaction of ferulic acid to maintain the titer of ferulic acid despite long-term storage and prevent discoloration and a reduction in efficacy.

In addition, it is possible to improve the solubility of ferulic acid to overcome the problem of precipitation and prevent skin irritation.

### DESCRIPTION OF DRAWINGS

FIG. 1 shows photographs of the appearance of compositions according to Experimental Example 1.

FIG. 2 shows photographs of the appearance of the composition of Example 3 in Experimental Example 5 at week 4.

FIG. 3 shows photographs of the appearance of each composition of Example 6 and Comparative Example 19 in Experimental Example 6 after 6-month storage at 25° C.

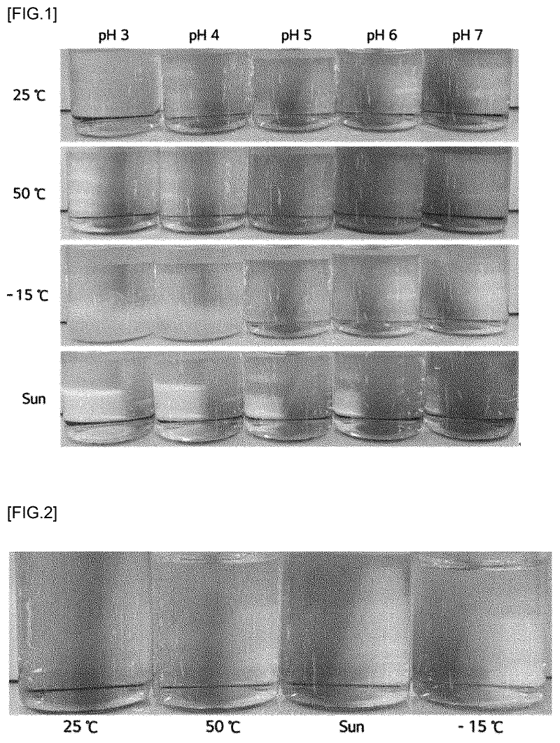

### MODES OF THE INVENTION

Hereinafter, the present invention will be described in detail.

As the present invention is subject to various changes and can have various forms, the following specific embodiments and descriptions are only intended to help understanding of the present invention, and are not intended to limit the present invention to a specific disclosed form. The scope of the present invention should be understood to include all changes, equivalents, and substitutes included in the spirit and technical scope of the present invention.

In the present invention, “ferulic acid or 4-hydroxy-3-methoxycinnamic acid” is a phenolic compound and has the structure of Chemical Formula 1 below. Ferulic acid may have antioxidant, antibacterial, skin brightening effects due to inhibition of the regeneration of melanin pigment, UV protection, skin elasticity, and anti-aging effects.

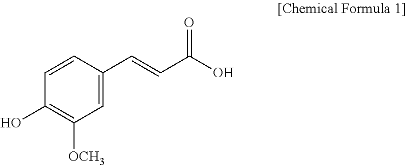 [SMILES: COc1cc(/C=C/C(=O)O)ccc1O]

The use of ferulic acid in cosmetics is limited because ferulic acid has very low solubility in water and is easily oxidized. Accordingly, the present invention is intended to provide a cosmetic composition with improved formulation stability of ferulic acid.

Ferulic acid begins to be decomposed by a decarboxylation reaction, which is accelerated at pH 5 or above. The decarboxylation reaction of ferulic acid is shown in Scheme 1 below.

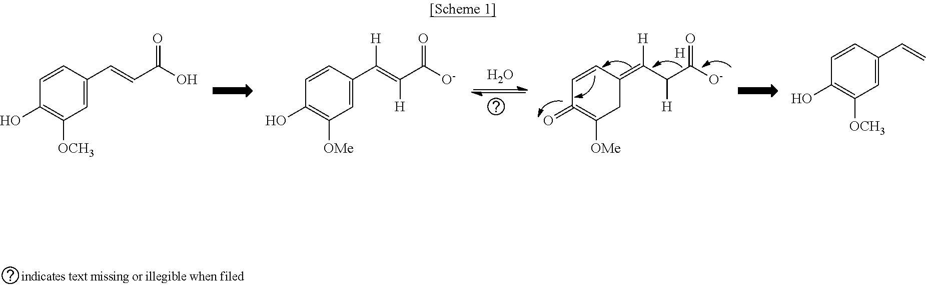

The decarboxylative decomposition reaction of ferulic acid occurs when the negatively charged carboxyl functional group of ferulic acid forms an ion-pair with a positive charge in an aqueous solution. It was expected that the decomposition of ferulic acid could be inhibited by inhibiting the interaction between charges, and to this end, a method to increase the energy required for the decarboxylative decomposition reaction was sought. Although the use of sodium or potassium ions as the positive charge was considered, it was not sufficient to improve the stability of ferulic acid because they were easily ionized in an aqueous solution and their binding energy was low. The inventors of the present invention made continuous attempts and completed the present invention after confirming that the stability of ferulic acid was improved when a basic amino acid was used as a cationic component.

Therefore, the present invention provides a cosmetic composition including ferulic acid; a basic amino acid; and a pH adjusting agent.

In an embodiment of the present invention, the content of the ferulic acid may be 0.1 wt % or more based on the total weight of the composition. Specifically, the content of the ferulic acid may be 0.1 to 10 wt %, more specifically, 0.2 wt % or more, 0.3 wt % or more, 0.5 wt % or more, 0.8 wt % or more, 1 wt % or more, or 2 wt % or more, and 8 wt % or less, 7 wt % or less, 6 wt % or less, or 5 wt % or less.

In an embodiment of the present invention, the basic amino acid may be any basic amino acid known to those skilled in the art, and may preferably be one or more types selected from the group consisting of arginine, lysine, histidine, and carnitine, their acids or bases, organic or mineral salts, and most preferably, carnitine or arginine. Basic amino acids are harmless to the human body and do not cause skin irritation even when used in large amounts, and they may improve the formulation stability of ferulic acid. In particular, apart from basic amino acids, the use of other components having amine groups, such as triethanolamine (TEA) or tromethamine (TRIS), may be considered for the same purpose, but triethanolamine and tromethamine are dangerous due to unreacted amines (N-nitrosodiethanolamine (NDELA) in TEA and secondary amines in TRIS), and even when these unreacted amines are present at a non-hazardous level, they may cause skin irritation. Therefore, basic amino acids are more preferable.

The content of the basic amino acid may vary depending on the content of ferulic acid and a pH adjusting agent and the pH of the composition, and is not limited as long as it reaches the pH range of the present invention when included with ferulic acid and the pH adjusting agent in the composition.

In an embodiment of the present invention, the content of the basic amino acid may be 0.001 to 10 wt % based on the total weight of the composition. The content of the basic amino acid may preferably be 0.001 to 5 wt %, 0.01 to 3 wt %, or 0.01 to 1 wt %. When the basic amino acid is used in the above content range, the decarboxylative decomposition reaction of ferulic acid may be sufficiently inhibited, but when the basic amino acid is used in an excessive amount, skin irritation may be caused.

The composition may have a pH of 4.0 to 6.0. More specifically, the pH may be 4.4 to 6.0, 4.4 to 5.7, more preferably, 4.4 to 5.5, 4.4 to 5.3, or 4.4 to 5.2. The pH may be adjusted by a pH adjusting agent. As the pH rises, the solubility of ferulic acid is improved, and thus precipitation may be reduced, but the decomposition of ferulic acid and the titer reduction may be accelerated. Thus, the pH needs to be adjusted within an appropriate range.

The pH adjusting agent is a component that is used to minimize the pH change and maintain it within a certain range through a conjugate base/acid reaction rather than simply increasing or decreasing the pH like strong acids and strong bases. The pH adjusting agent may be a pH buffer.

Specifically, the pH adjusting agent may be one or more selected from the group consisting of sodium citrate, potassium citrate, magnesium citrate, salicylic acid, glutamic acid, sodium phosphate. disodium phosphate, potassium phosphate, dipotassium phosphate, calcium phosphate, lactic acid, sulfuric acid, succinic acid, sodium lactate, potassium lactate, calcium lactate, and magnesium lactate. Most preferably, the pH adjusting agent may be sodium citrate.

The pH adjusting agent serves to adjust the pH of the composition and may inhibit the decarboxylative decomposition reaction of ferulic acid, like a basic amino acid, through the interaction of the functional group of the pH adjusting agent, such as carboxyl, hydroxyl, or amine groups, with ferulic acid (for example, hydrogen bonding or the van der Waals force).

In addition, the pH adjusting agent may further include EDTA.

The content of the pH adjusting agent may vary depending on the content of ferulic acid and a basic amino acid and the pH range of the composition, and is not limited as long as it reaches the pH range of the present invention when included with ferulic acid and the basic amino acid in the composition.

In an embodiment of the present invention, the content of the pH adjusting agent may be 0.001 to 3 wt % based on the total weight of the composition. More specifically, the content of the pH adjusting agent may be 0.01 to 3 wt %, 0.1 to 2 wt %, or 0.15 to 1.5 wt %. When the pH adjusting agent is included in an amount less than the above content, it is impossible to inhibit the pH increase caused by the decomposition of ferulic acid, which may accelerate the decomposition of ferulic acid and the titer reduction, and when the pH adjusting agent is included in an amount more than the above content, product quality may be affected.

In the present invention, both the pH adjusting agent and the basic amino acid may serve as a pH buffer and may reduce the impact of external changes on ferulic acid compared to when only one of them is used.

In an embodiment of the present invention, the weight ratio of ferulic acid, the basic amino acid, and the pH adjusting agent may be 1:0.1 to 1.5:0.1 to 1.

The composition of the present invention may further include water and/or polyol.

In an embodiment of the present invention, in order to avoid a sticky or heavy feeling of use, the content of the polyol may be 20 wt % or less or 17.5 wt % or less based on the total weight of the composition. Preferably, the content of the polyol may be 1 to 20 wt %, 2 to 18 wt %, or 5 to 17.5 wt %.

The polyol may include glycerin and one or more types of glycols, and the glycols may be one or more types selected from the group consisting of dipropylene glycol, butylene glycol, propanediol, ethoxydiglycol, 2,3-butanediol, and 1,2-hexanediol. Preferably, in consideration of solubility, the polyol may be propanediol and/or ethoxydiglycol.

The polyol may serve as a moisturizer, and a sufficient amount of polyol may be used to stabilize ferulic acid in the formulation. Otherwise, ferulic acid may precipitate. In the present invention, the amount of polyol may be about 0.01 to 50 times, 0.05 to 30 times, 0.1 to 20 times, or 3 to 20 times, that of ferulic acid. When the polyol content is too high, skin irritation may be caused.

In an embodiment of the present invention, the content of water may be 50 wt % or more. Specifically, the content of water may be 50 to 90 wt %, 50 to 80 wt %, or 60 to 80 wt %.

In an embodiment of the present invention, the cosmetic composition of the present invention may not discolor or precipitate or may maintain its titer even when stored at 50° C. for 4 weeks.

In the present invention, “maintaining the titer” means that ferulic acid is hardly decomposed even when stored at 50° C. for 4 weeks, and specifically, it may mean that the decomposition degree of ferulic acid is 10% or less, 5% or less, or ferulic acid is not decomposed at all.

The present invention also provides an external preparation for skin including the above-described cosmetic composition.

The components included in the cosmetic composition of the present invention are active ingredients, and in addition to the active ingredients, components commonly used in cosmetic compositions, such as conventional auxiliaries including antioxidants, stabilizers, solubilizers, vitamins, ultraviolet absorbers, preservatives, pH adjusting agents, colorants, pigments and fragrances, and carriers, may be included.

The cosmetic composition of the present invention may be prepared in any formulation commonly prepared in the art, and it may be formulated as a solution, a suspension, an emulsion, paste, a gel, a cream, a lotion, powder, soap, a cleansing oil, a powder foundation, an emulsion foundation, a wax foundation, a pack, a massage cream, and a spray, but is not limited thereto. More specifically, it may be prepared in the form of a toner, a lotion, a softening lotion, a nourishing lotion, a nourishing cream, a massage cream, an essence, an eye cream, a cleansing cream, a cleansing foam, a cleansing water, a pack, a spray, or powder.

Each of the above-mentioned components contained in the cosmetic composition according to the present invention may be included in the cosmetic composition of the present invention within a range that does not exceed the maximum usage amount specified in the regulations related to “Use/Permission on Cosmetics” stipulated by each government around the world.

Another aspect of the present invention provides a method of preparing a cosmetic composition including ferulic acid, which includes preparing a cosmetic composition with a pH of 4.0 to 6.0 by mixing ferulic acid with a basic amino acid and a pH adjusting agent.

In the preparation method, the content of ferulic acid may be 0.1 wt % or more based on the total weight of the composition.

In the preparation method, polyol and/or water may be additionally mixed.

The preparation method may further include mixing the basic amino acid and the pH adjusting agent and heating the mixture to 40 to 70° C.

The preparation method may be intended to prepare a cosmetic composition substantially without precipitation, discoloration, and/or a reduction in the titer of ferulic acid. Specifically, the preparation method may be intended to prepare a cosmetic composition substantially without precipitation, discoloration, and/or a reduction in the titer of ferulic acid even after storage at 50° C. for 4 weeks.

In the preparation method, the content of the above-described ferulic acid, basic amino acid, pH adjusting agent, and cosmetic composition may be applied as is.

Still another aspect of the present invention provides a use of a basic amino acid and a pH adjusting agent for the preparation of a cosmetic composition including ferulic acid substantially without precipitation, discoloration, and/or a reduction in the titer of ferulic acid.

In the above use, the content of the above-described ferulic acid, basic amino acid, pH adjusting agent, and cosmetic composition may be applied as is.

In the present invention, “substantially without” means that no precipitation, discoloration, and/or titer reduction of ferulic acid occur even when ferulic acid is stored at 50° C. for 4 weeks, or that the decomposition degree of ferulic acid is 10% or less or 5% or less.

Hereinafter, the present invention will be described in detail through the following experimental examples. The following experimental examples are intended to illustrate the present invention, and the content of the present invention is not limited thereby. In addition, since these experimental examples are only intended to help understand the present invention, the scope of the present invention is not limited by them in any way.

### EXAMPLE

In this example, the stabilities of ferulic acid according to pH (Experimental Example 1), cationic components used to stabilize ferulic acid (Experimental Example 2), the type of amino acids (Experimental Example 3), and the content of a pH adjusting agent (Experimental Example 4) were confirmed, and as in Experimental Examples 5 and 6, the composition according to the present invention was prepared and the formulation stability and the titer were analyzed.

### <Formulation Stability Evaluation>

For the compositions prepared in each experiment below, the stability of the formulation was confirmed. Specifically, the compositions prepared in each experiment were stored at 25° C., 50° C., and −15° C. for 4 weeks or stored for 8 hours under exposure conditions, that is, exposure to sunlight, and then discoloration and precipitation were evaluated based on the following criteria.

### 1. Discoloration

O: No color change or difficult to notice
Δ: Noticeable color change but not affecting preference
X: Severe color change enough to adversely affect preference

### 2. Precipitation

O: No precipitation
X: Precipitation occurs

### Experimental Example 1. Observation of Stability of Ferulic Acid According to pH

According to the compositions in Table 1 below, the components were heated and mixed at 50° C. and then cooled to prepare the compositions of Comparative Examples 1 to 5.

TABLE 1

| | Comparative | Comparative | Comparative | Comparative | Comparative |
|---|---|---|---|---|---|
| | Example 1 | Example 2 | Example 3 | Example 4 | Example 5 |
| Water | Remainder | Remainder | Remainder | Remainder | Remainder |
| Ferulic acid | 0.3 | 0.3 | 0.3 | 0.3 | 0.3 |
| Ethoxydiglycol | 2.5 | 2.5 | 2.5 | 2.5 | 2.5 |
| 2,3-Butanediol | 10 | 10 | 10 | 10 | 10 |
| 1,2-Hexanediol | 2 | 2 | 2 | 2 | 2 |
| Tromethamine | — | 0.05 | 0.2 | 0.3 | 0.4 |
| pH | 3 | 4 | 5 | 6 | 7 |

The <Formulation stability evaluation> was performed on the compositions of Comparative Examples 1 to 5 prepared above, and the results are shown in Table 2 and Table 3.

TABLE 2

| Discoloration | Comparative | Comparative | Comparative | Comparative | Comparative |
|---|---|---|---|---|---|
| evaluation | Example 1 | Example 2 | Example 3 | Example 4 | Example 5 |
| 25° C. | ◯ | ◯ | ◯ | ◯ | Δ |
| 50° C. | ◯ | Δ | Δ | X | X |
| −15° C. | ◯ | ◯ | ◯ | ◯ | ◯ |
| Exposure to | ◯ | ◯ | ◯ | Δ | X |
| sunlight | | | | | |

TABLE 3

| Precipitation | Comparative | Comparative | Comparative | Comparative | Comparative |
|---|---|---|---|---|---|
| evaluation | Example 1 | Example 2 | Example 3 | Example 4 | Example 5 |
| 25° C. | X | ◯ | ◯ | ◯ | ◯ |
| 50° C. | ◯ | ◯ | ◯ | ◯ | ◯ |
| −15° C. | X | X | ◯ | ◯ | ◯ |
| Exposure to | ◯ | ◯ | ◯ | ◯ | ◯ |
| sunlight | | | | | |

Referring to Table 3, it was confirmed that when the pH was low, precipitation occurred under freezing conditions (Comparative Examples 1 and 2), and referring to Table 2, it was confirmed that when the pH was high, discoloration occurred under exposure conditions, that is, exposure to a high temperature or light (Comparative Examples 2 to 5). In other words, it can be seen that it is necessary to inhibit the decomposition of ferulic acid because the decomposition of ferulic acid is accelerated over time when the pH is 4 or above.

### Experimental Example 2. Observation of Stability of Ferulic Acid According to Cationic Components

According to the compositions in Table 4 below, the components were heated and mixed at 50° C. and then cooled to prepare the compositions of Comparative Examples 6 to 9. Arginine, tromethamine, NaOH, and KOH were added in appropriate amounts to adjust the pH to 5.

TABLE 4

| | Comparative | Comparative | Comparative | Comparative |
|---|---|---|---|---|
| | Example 6 | Example 7 | Example 8 | Example 9 |
| Water | Remainder | Remainder | Remainder | Remainder |
| Ferulic acid | 0.5 | 0.5 | 0.5 | 0.5 |
| Ethoxydiglycol | 2.5 | 2.5 | 2.5 | 2.5 |
| 2,3-Butanediol | 10 | 10 | 10 | 10 |
| 1,2-Hexanediol | 2 | 2 | 2 | 2 |
| Arginine | Appropriate | — | — | — |
| | amount | | | |
| Tromethamine | — | Appropriate | — | — |
| | | amount | | |
| NaOH | — | — | Appropriate | — |
| | | | amount | |
| KOH | — | — | — | Appropriate |
| | | | | amount |
| pH | 5 | 5 | 5 | 5 |

The <Formulation stability evaluation> was performed on the compositions of Comparative Examples 6 to 9 prepared above, and the results are shown in Table 5 and Table 6.

TABLE 5

| Discoloration | Comparative | Comparative | Comparative | Comparative |
|---|---|---|---|---|
| evaluation | Example 6 | Example 7 | Example 8 | Example 9 |
| 25° C. | ◯ | ◯ | ◯ | ◯ |
| 50° C. | Δ | Δ | Δ | Δ |
| −15° C. | ◯ | ◯ | ◯ | ◯ |
| Exposure to | ◯ | ◯ | ◯ | ◯ |
| sunlight | | | | |

TABLE 6

| Precipitation | Comparative | Comparative | Comparative | Comparative |
|---|---|---|---|---|
| evaluation | Example 6 | Example 7 | Example 8 | Example 9 |
| 25° C. | ◯ | ◯ | ◯ | ◯ |
| 50° C. | ◯ | ◯ | ◯ | ◯ |
| −15° C. | ◯ | ◯ | ◯ | ◯ |
| Exposure to | ◯ | ◯ | ◯ | ◯ |
| sunlight | | | | |

As shown in Table 5 and Table 6, there was no significant difference in the stability of ferulic acid in Comparative Examples 6 to 9. However, it was confirmed that slight discoloration occurred at a high temperature (4 weeks at 50° C.). In addition, it was confirmed that in Comparative Examples 6 to 9, no precipitation occurred under freezing conditions at pH 5, which was similar to Comparative Example 3. Accordingly, it was confirmed that as a component for stabilizing ferulic acid, basic amino acids can be used instead of other harmful components such as tromethamine and strong bases, KOH and NaOH.

### Experimental Example 3. Observation of Stability of Ferulic Acid According to Amino Acid Type

According to the compositions in Table 7 below, the components were heated and mixed at 50° C. and then cooled to prepare the compositions of Comparative Examples 10 to 13.

TABLE 7

| | Comparative | Comparative | Comparative | Comparative |
|---|---|---|---|---|
| | Example 10 | Example 11 | Example 12 | Example 13 |
| Water | Remainder | Remainder | Remainder | Remainder |
| Ferulic acid | 0.5 | 0.5 | 0.5 | 0.5 |
| Ethoxydiglycol | 2.5 | 2.5 | 2.5 | 2.5 |
| 2,3-Butanediol | 10 | 10 | 10 | 10 |
| 1,2-Hexanediol | 2 | 2 | 2 | 2 |
| Arginine | Appropriate | — | — | — |
| | amount | | | |
| Carnitine | — | Appropriate | — | — |
| | | amount | | |
| Aspartic acid | — | — | Appropriate | — |
| | | | amount | |
| Serine | — | — | — | Appropriate |
| | | | | amount |
| pH | 5 | 5 | 3 | 4 |

The <Formulation stability evaluation> was performed on the compositions of Comparative Examples 10 to 13 prepared above, and the results are shown in Table 8 and Table 9.

TABLE 8

| | Comparative | Comparative | Comparative | Comparative |
|---|---|---|---|---|
| Discoloration | Example 10 | Example 11 | Example 12 | Example 13 |
| 25° C. | ◯ | ◯ | — | ◯ |
| 50° C. | Δ | Δ | — | ◯ |
| −15° C. | ◯ | ◯ | — | ◯ |
| Exposure to | ◯ | ◯ | — | ◯ |
| sunlight | | | | |

TABLE 9

| | Comparative | Comparative | Comparative | Comparative |
|---|---|---|---|---|
| Precipitation | Example 10 | Example 11 | Example 12 | Example 13 |
| 25° C. | ◯ | ◯ | — | X |
| 50° C. | ◯ | ◯ | — | ◯ |
| −15° C. | ◯ | ◯ | — | X |
| Exposure to | ◯ | ◯ | — | ◯ |
| sunlight | | | | |

As shown in Table 8 and Table 9, it was confirmed that Comparative Examples 10 and 11 respectively using arginine and carnitine among basic amino acids had excellent formulation stability. In the case of Comparative Example 12 using aspartic acid, one of the acidic amino acids, since aspartic acid did not dissolve during the preparation process, its contribution to the stabilization of ferulic acid could not be confirmed. In the case of Comparative Example 13 using serine, one of the neutral amino acids, the pH did not increase to 4 or above even when an excessive amount was added, and thus precipitation occurred.

### Experimental Example 4. Observation of Stability According to pH Adjusting Agent Content

According to the compositions in Table 10 below, the components were heated and mixed at 50° C. and then cooled to prepare the compositions of Comparative Examples 14 to 18.

TABLE 10

| | Comparative | Comparative | Comparative | Comparative | Comparative |
|---|---|---|---|---|---|
| | Example 14 | Example 15 | Example 16 | Example 17 | Example 18 |
| Water | Remainder | Remainder | Remainder | Remainder | Remainder |
| Ferulic acid | 0.5 | 0.5 | 0.5 | 0.5 | 0.5 |
| Ethoxydiglycol | 2.5 | 2.5 | 2.5 | 2.5 | 2.5 |
| 2,3-Butanediol | 10 | 10 | 10 | 10 | 10 |
| 1,2-Hexanediol | 2 | 2 | 2 | 2 | 2 |
| Sodium citrate | 0.1 | 0.2 | 0.5 | 0.8 | 1.0 |
| pH | 4.3 | 4.6 | 5.2 | 5.5 | 5.7 |

The <Formulation stability evaluation> was performed on the compositions of Comparative Examples 14 to 18 prepared above, and the results are shown in Table 11 and Table 12.

TABLE 11

| Discoloration | Comparative | Comparative | Comparative | Comparative | Comparative |
|---|---|---|---|---|---|
| evaluation | Example 14 | Example 15 | Example 16 | Example 17 | Example 18 |
| 25° C. | ◯ | ◯ | ◯ | ◯ | ◯ |
| 50° C. | ◯ | ◯ | ◯ | ◯ | Δ |
| −15° C. | ◯ | ◯ | ◯ | ◯ | ◯ |
| Exposure to | ◯ | ◯ | Δ | Δ | Δ |
| sunlight | | | | | |

TABLE 12

| Precipitation | Comparative | Comparative | Comparative | Comparative | Comparative |
|---|---|---|---|---|---|
| evaluation | Example 14 | Example 15 | Example 16 | Example 17 | Example 18 |
| 25° C. | ◯ | ◯ | ◯ | ◯ | ◯ |
| 50° C. | ◯ | ◯ | ◯ | ◯ | ◯ |
| −15° C. | X | X | ◯ | ◯ | ◯ |
| Exposure to | ◯ | ◯ | ◯ | ◯ | ◯ |
| sunlight | | | | | |

In Comparative Examples 14 and 15 which had a pH of 4.3 and 4.6 by including 0.1 wt % and 0.2 wt % of sodium citrate, respectively, ferulic acid precipitated under freezing conditions, whereas Comparative Examples 16 to 18 in which the pH was adjusted to 5.2 to 5.7 by including 0.5 wt %, 0.8 wt %, and 1.0 wt % of sodium citrate, respectively, were confirmed to be stable under freezing conditions. However, in Comparative Examples 16 to 18, slight discoloration occurred under sunlight exposure conditions.

### Experimental Example 5. Observation of Stability of Compositions According to the Present Invention

According to the compositions in Table 13 below, the components were heated and mixed at 50° C. and then cooled to prepare the compositions of Examples 1 to 5.

TABLE 13

| | Example 1 | Example 2 | Example 3 | Example 4 | Example 5 |
|---|---|---|---|---|---|
| Water | Remainder | Remainder | Remainder | Remainder | Remainder |
| Ferulic acid | 0.5 | 0.5 | 0.5 | 0.5 | 0.5 |
| Ethoxydiglycol | 2.5 | 2.5 | 2.5 | 2.5 | 2.5 |
| 2,3-Butanediol | 10 | 10 | 10 | 10 | 10 |
| 1,2-Hexanediol | 2 | 2 | 2 | 2 | 2 |
| Carnitine | 1.2 | 1.2 | 1.2 | 1.2 | 1.2 |
| Sodium citrate | 0.1 | 0.3 | 0.5 | 0.8 | 1.0 |
| pH | 4.4 | 4.9 | 5.2 | 5.5 | 5.7 |

The <Formulation stability evaluation> was performed on the compositions of Examples 1 to 5 prepared above, and the results are shown in Table 14 and Table 15.

TABLE 14

| Discoloration | Exam- | Exam- | Exam- | Exam- | Exam- |
|---|---|---|---|---|---|
| evaluation | ple 1 | ple 2 | ple 3 | ple 4 | ple 5 |
| 25° C. | ◯ | ◯ | ◯ | ◯ | ◯ |
| 50° C. | ◯ | ◯ | ◯ | ◯ | ◯ |
| −15° C. | ◯ | ◯ | ◯ | ◯ | ◯ |
| Exposure to | ◯ | ◯ | ◯ | ◯ | ◯ |
| sunlight | | | | | |

TABLE 15

| Precipitation | | | | | |
|---|---|---|---|---|---|
| evaluation | Example 1 | Example 2 | Example 3 | Example 4 | Example 5 |
| 25° C. | ◯ | ◯ | ◯ | ◯ | ◯ |
| 50° C. | ◯ | ◯ | ◯ | ◯ | ◯ |
| −15° C. | ◯ | ◯ | ◯ | ◯ | ◯ |
| Exposure to | ◯ | ◯ | ◯ | ◯ | ◯ |
| sunlight | | | | | |

Based on the results according to the amine component and the pH adjusting agent confirmed in Experimental Examples 1 to 4, the compositions of the example of the present invention were prepared, and it was confirmed that despite a slight difference in stability depending on the pH, the compositions had excellent formulation stability.

### Experimental Example 6. Observation of Stability of Emulsion Formulation

According to Table 16 below, water phase materials (raw materials 1 to 9) were mixed and heated to 50° C., and polymer phase materials (raw materials 10 to 12) were mixed and maintained at 50° C. Additionally, oil phase materials (raw materials 13 and 14) were mixed and heated to 50° C. The water phase and the polymer phase were mixed at 5,000 rpm for 5 minutes using a homomixer, and then the oil phase was added and stirred at 5,000 rpm for another 5 minutes. Afterward, the mixture was cooled to 30° C.

TABLE 16

| | | | Comparative |
|---|---|---|---|
| | | Example 6 | Example 19 |
| 1 | Water | Remainder | Remainder |
| 2 | Propanediol | 10 | 10 |
| 3 | Ethoxydiglycol | 2 | 2 |
| 4 | Glycerin | 4 | 4 |
| 5 | 1,2-Hexanediol | 1.5 | 1.5 |
| 6 | EDTA | 0.02 | 0.02 |
| 7 | Carnitine | 1.2 | — |
| 8 | Sodium citrate | 0.2 | — |
| 9 | Ferulic acid | 0.5 | 0.5 |
| 10 | Water | Small amount | Small amount |
| 11 | Polyacrylate crosspolymer-6 | 1 | 1 |
| 12 | Xanthan gum | 0.2 | 0.2 |
| 13 | Dimethicone | 10 | 10 |
| 14 | ABIL CARE XL 80* | 1 | 1 |
| | pH | 5.3 | 3.2 |

*ABIL CARE XL 80: Bis-PEG/PPG-20/5PEG/PPG-20/5 dimethicone, methoxy PEG/PPG-25/4 dimethicone

The compositions prepared according to Example 6 and Comparative Example 19 were evaluated in the same manner as the <Formulation stability evaluation>, except that discoloration was confirmed after 6-month storage for the evaluation at temperatures of 25° C., 50° C., and −15° C., and the results are shown in Table 17.

TABLE 17

| Discoloration | | Comparative |
|---|---|---|
| evaluation | Example 6 | Example 19 |
| 25° C. | ◯ | X |
| 50° C. | ◯ | X |
| −15° C. | ◯ | ◯ |
| Exposure to | ◯ | X |
| sunlight | | |

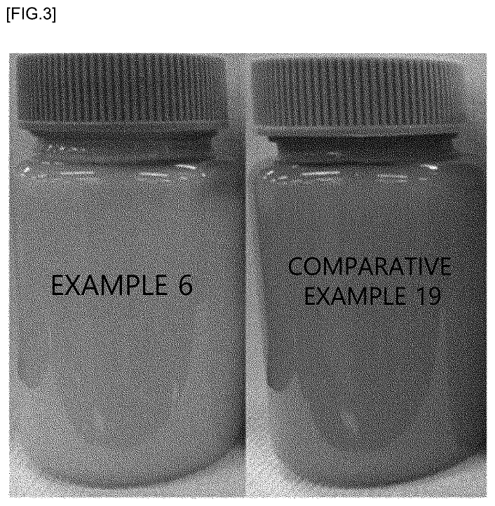

It was confirmed that the formulation stability of the composition of Example 6 was superior to that of Comparative Example 19. In particular, as shown in FIG. 3, there was a clear difference in color when the composition was stored at 25° C. for 6 months, which can directly affect the quality of products.

### Experimental Example 7. Titer Analysis of Ferulic Acid According to pH

The reduction rate of ferulic acid was confirmed when Comparative Examples 1, 3, and 5 prepared in Experimental Example 1 and Example 3 prepared in Experimental Example 5 were stored at 50° C. for 4 weeks. In other words, the amount of ferulic acid at the time of measurement compared to the initial content of ferulic acid was quantified to measure the amount of reduced ferulic acid. The initial value of ferulic acid in Comparative Examples 1, 3, and 5 was 0.3 wt % and in Example 3 was 0.5 wt %.

TABLE 18

| | 50° C. for | Reduction | |
|---|---|---|---|
| | 4 weeks (wt %) | rate (%) | pH |
| Comparative Example 1 | 0.24 | 17 | 3.0 |
| Comparative Example 3 | 0.23 | 20 | 5.0 |
| Comparative Example 5 | 0.21 | 27 | 7.0 |
| Example 3 | 0.50 | 0 | 5.2 |

In Comparative Examples 1, 3, and 5, ferulic acid was decomposed when stored at 50° C. for 4 weeks, and it was confirmed that the titer was significantly reduced as the pH increased.

In addition, in Example 3, the pH was 5, which was the same as Comparative Example 3, but ferulic acid was hardly decomposed so the content of ferulic acid was 0.50 wt %. Therefore, it was confirmed that the titer was maintained stably without reduction at a high temperature.

For Example 6 and Comparative Example 19 which were the emulsion formulations prepared in Experimental Example 6, the reduction rate of ferulic acid was confirmed, and the results are shown in Table 19.

TABLE 19

| | 50° C. for | Reduction | |
|---|---|---|---|
| | 4 weeks (wt %) | rate (%) | pH |
| Example 6 | 0.50 | 0 | 5.3 |
| Comparative Example 19 | 0.43 | 14 | 3.2 |

After confirming the residual ferulic acid at a high temperature over time, it can be seen that ferulic acid was stable without decomposition in Example 6, whereas in Comparative Example 19, ferulic acid was decomposed.

1. A cosmetic composition comprising:
ferulic acid;
a basic amino acid; and
a pH adjusting agent,
wherein the basic amino acid is one or more selected from the group consisting of lysine, histidine, and carnitine, and
wherein the content of the ferulic acid is 0.1 wt % or more based on the total weight of the composition and the composition has a pH of 4.0 to 6.0.

2. (canceled)

3. The composition of claim 1, wherein the content of the basic amino acid is 0.001 to 10 wt % based on the total weight of the composition.

4. The composition of claim 1, wherein the pH adjusting agent is one or more selected from the group consisting of sodium citrate, potassium citrate, magnesium citrate, salicylic acid, glutamic acid, sodium phosphate. disodium phosphate, potassium phosphate, dipotassium phosphate, calcium phosphate, lactic acid, sulfuric acid, succinic acid, sodium lactate, potassium lactate, calcium lactate, and magnesium lactate.

5. The composition of claim 1, wherein the content of the pH adjusting agent is 0.001 to 3 wt % based on the total weight of the composition.

6. The composition of claim 1, further comprising one or more polyols.

7. The composition of claim 6, wherein the polyol is one or more selected from the group consisting of glycerin, dipropylene glycol, butylene glycol, propanediol, ethoxydiglycol, 2,3-butanediol, and 1,2-hexanediol.

8. The composition of claim 1, wherein water is included in an amount of 50 wt % or more based on the total weight of the composition.

9. The composition of claim 1, wherein no discoloration and precipitation occur and the titer is maintained even when the composition is stored at 50° C. for 4 weeks.

10. An external preparation for skin comprising the cosmetic composition according to claim 1.

11. A method of preparing a cosmetic composition including ferulic acid, the method comprising preparing a cosmetic composition with a pH of 4.0 to 6.0 by mixing ferulic acid with a basic amino acid and a pH adjusting agent,
wherein the basic amino acid is one or more selected from the group consisting of lysine, histidine, and carnitine.

12. The method of claim 11, wherein the preparation method is for preparing a cosmetic composition substantially without the precipitation, discoloration, and/or a reduction in the titer of ferulic acid.

13. An external preparation for skin comprising the cosmetic composition according to claim 3.

14. An external preparation for skin comprising the cosmetic composition according to claim 4.

15. An external preparation for skin comprising the cosmetic composition according to claim 5.

16. An external preparation for skin comprising the cosmetic composition according to claim 6.

17. An external preparation for skin comprising the cosmetic composition according to claim 7.

18. An external preparation for skin comprising the cosmetic composition according to claim 8.

19. An external preparation for skin comprising the cosmetic composition according to claim 9.
